(19) United States
(12) Patent Application Publication
(10) Pub. No.: US 2022/0277840 A1
(43) Pub. Date: Sep. 1, 2022

(54) SYSTEMS, MEDIA, AND METHODS FOR MEASURING HEALTH CARE PROVIDER PERFORMANCE AND TO OPTIMIZE PROVISION OF HEALTH CARE SERVICES

(71) Applicant: CareJourney, Arlington, VA (US)

(72) Inventors: Chris Lester, Arlington, VA (US); Elena Kuenzel, Arlington, VA (US); Chris Freyder, Arlington, VA (US); Dan Ross, Arlington, VA (US); Aneesh Chopra, Arlington, VA (US)

(73) Assignee: CareJourney, Arlington, VA (US)

(21) Appl. No.: 17/631,644

(22) PCT Filed: Jul. 30, 2020
(86) PCT No.: PCT/US2020/044261

§ 371 Date: Jan. 31, 2022

Related U.S. Application Data
(60) Provisional application No. 62/880,523, filed on Jul. 30, 2019.

(51) Int. Cl. G16H 40/20; G06Q 10/06

(52) U.S. Cl. CPC G16H 40/20; G06Q 10/06393

(57) ABSTRACT

Systems, methods, and media for measuring health care provider performance and optimizing provision of health care services are provided. A method may include identifying potential chronic conditions a patient has based on a chronic conditions mapping. The method may also comprise determining a cost score. The method may further comprise determining an outcome score. The method may additionally comprise obtaining a final outcome score for each of a plurality of providers based on a variance weighted average of probabilities for groups of metrics pertaining to each provider. The method additionally include generating a final quality index based upon the final outcome score for each provider.

### CROSS REFERENCE TO RELATED APPLICATION

This application is a National Phase Application of International Application No. PCT/US2020/044261, filed Jul. 30, 2020 and claims the benefit of U.S. Provisional Patent Application Ser. No. 62/880,523 filed Jul. 30, 2019, which is incorporated by reference herein in its entirety.

### TECHNICAL FIELD

The present specification generally relates to data assessment systems, and more particularly, assessment systems for evaluating performance of healthcare providers.

### BACKGROUND

Tactics for evaluating healthcare providers vary widely, from online forums comprising personal feedback testimonials from individual patients for a particular healthcare service provider, to rating systems using various data for ranking medical institutions such as hospitals and other healthcare facilities. The various tactics incorporate numerous methodologies in evaluating performance of professional healthcare service providers such that the industry is devoid of a consistent assessment standard. Additionally, various tactics incorporate datasets in a partial, incomplete, or biased approach such that the resulting assessments might not be impartial, thereby minimizing an overall reliability of the evaluation shortcomings scheme.

Accordingly, a need exists for a comprehensive assessment systems, media, and methods for generating data-driven evaluations of healthcare service providers.

### SUMMARY

A method may comprise identifying potential chronic conditions a patient has based on a chronic conditions mapping. The method may also comprise determining a cost score. The method may further comprise determining an outcome score. The method may additionally comprise obtaining a final outcome score for each of a plurality of providers based on a variance weighted average of probabilities for groups of metrics pertaining to each provider. The method may also further comprise generating a final quality index based upon the final outcome score for each provider.

In another embodiment, a system may comprise memory and a processor coupled to the memory, wherein the processor is configured to identify potential chronic conditions a patient has based on a chronic conditions mapping. The processor may be further configured to determine a cost score. The processor may also be configured to determine an outcome score. The processor may be additionally configured to obtain a final outcome score for each of a plurality of providers based on a variance weighted average of probabilities for groups of metrics pertaining to each provider. The processor also may be configured to generate a final quality index based upon the final outcome score for each provider.

In yet another embodiment, a non-transitory computer readable medium embodies computer-executable instructions that, when executed by a processor, cause the processor to cute operations comprising identifying potential chronic conditions a patient has based on a chronic conditions mapping. The operations may further comprise determining a cost score. The operations may also comprise determining an outcome score. The operations may additionally comprise obtaining a final outcome score for each of a plurality of providers based on a variance weighted average of probabilities for groups of metrics pertaining to each provider. The operations may still further comprise generating a final quality index based upon the final outcome score for each provider.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary, and are not intended to limit the subject matter defined by the claims. The following detailed description of the illustrative embodiments can be understood when read in conjunction with the following drawings, where like structure is indicated with like reference numerals and in which:

FIG. 1 schematically depicts an illustrative example of an index assessment system according to one or more embodiments shown or described herein;

FIG. 2 schematically depicts the index assessment system of FIG. 1 according to one or more embodiments shown or described herein;

FIG. 3 schematically depicts a table of statistics size among payers in 2018 according to one or more embodiments shown or described herein;

FIG. 4 schematically depicts a table of breakout cost and outcome scores for two exemplary service providers according to one or more embodiments shown or described herein;

FIG. 5 schematically depicts a table of total nationwide primary care physicians and specialist coverage scores using the index assessment system of FIG. 1 according to one or more embodiments shown or described herein;

FIG. 6 schematically depicts a table of Accountable Care Organizations (ACOS) with patients using high scoring specialists according to one or more embodiments shown or described herein;

FIG. 7 is a flow chart depicting acute episodes according to one or more embodiments shown and described herein;

FIG. 8 is a flow chart depicting chronic condition episodes according to one or more embodiments shown and described herein;

FIG. 9 is a flow chart depicting primary care physician (PCP) episodes according to one or more embodiments shown and described herein;

FIG. 10 is a flow chart depicting final physician scoring according to one or more embodiments shown and described herein; and

FIG. 11 is a block diagram illustrating computing hardware utilized in one or more devices, according one or more embodiments shown and described herein.

### DETAILED DESCRIPTION

Embodiments of the present disclosure are directed to methods, systems, and media for measuring health care provider performance and optimizing provision of health care services.

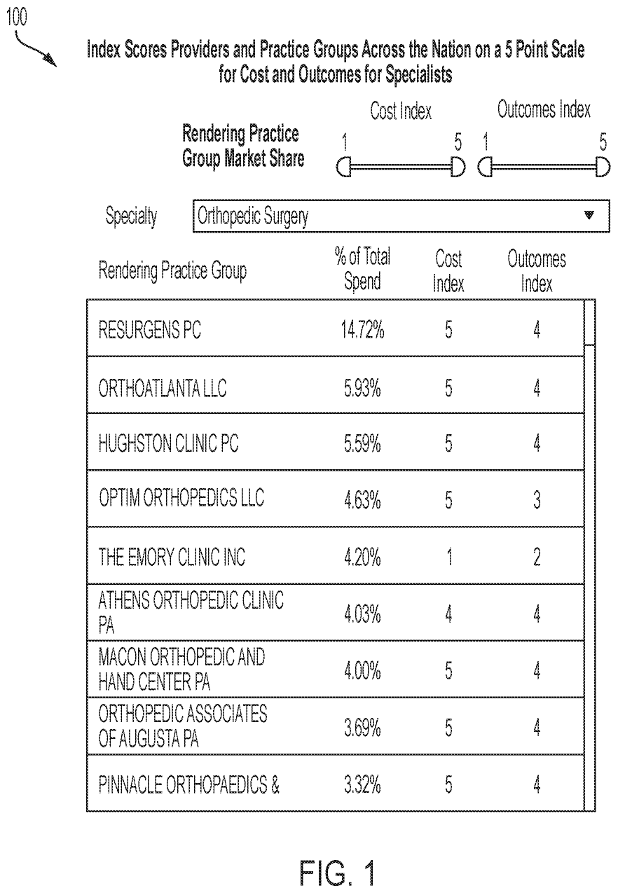

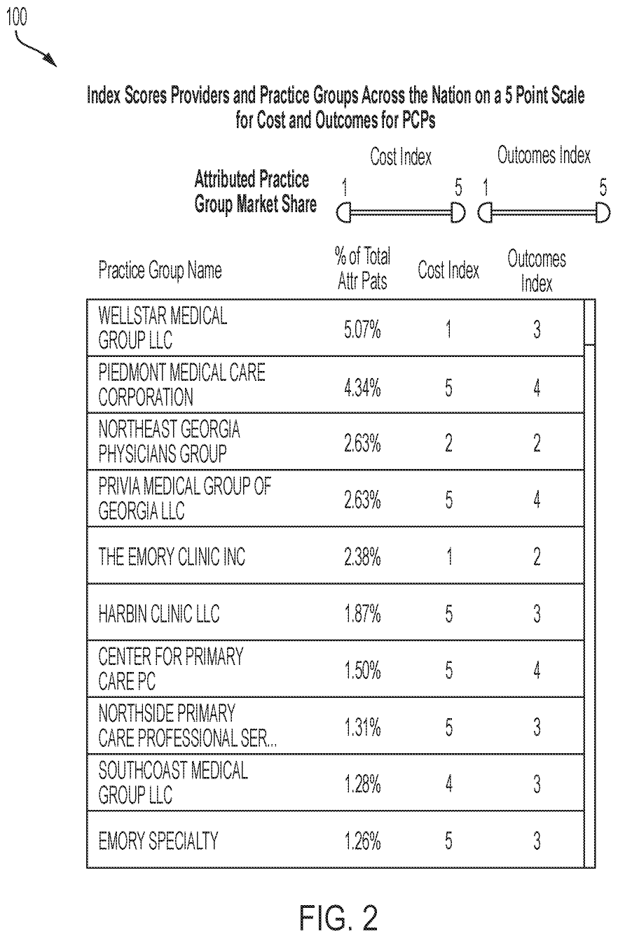

FIGS. 1-2 schematically depict an exemplary “Index Assessment System 100” that serves as a provider cost and rating system of healthcare service providers. The Index Assessment System 100 is based on access to the fully identifiable Medicare Fee-For-Service claims dataset. With the Index Assessment System 100, payers, hospitals, health systems, and Accountable Care Organizations (ACOS) can measure and score over 950,000 primary care physicians (PCPs), specialists, and surgeons across dozens of industry-accepted cost and outcome metrics using easy-to-understand cost and outcome scores. As described in greater detail herein, the Index Assessment System 100 may allow operators (e.g., customers) to easily and quickly benchmark providers by specialty and/or geography, such as a Core-Based Statistical Area (CB SA), in minutes, eliminating months of specialized work and the high cost normally associated with the performance of such a thorough and rigorous statistical analysis.

The Index Assessment System 100 may provide a comprehensive approach assessing both cost and outcomes from patient episodes, rather than simply assessing individual treatments or procedures. This system may create reliable benchmarks for more than 950,000 healthcare providers. Healthcare providers can be the source of much of the variation in cost and/or quality in healthcare. The Index Assessment System 100 may score providers based on cost and outcomes using open component algorithms to measure the effect of healthcare providers on the care of the patients they manage. The terms “patient” and “beneficiary” may be used interchangeably herein. Specialists and PCPs may be rated on two 5-point scales: one for cost-efficiency and one for outcomes, corresponding to quintiles of their performance against similar healthcare providers in their region. The Index Assessment System 100 may score providers and practice groups across the nation, by way of non-limiting example, on a five-point scale for cost and outcomes for specialists (FIG. 1) and PCPs (FIG. 2). The Index Assessment System 100 may be extended to score facilities as well.

The Index Assessment System 100 may include a calculation methodology that generally follows one or more steps for each healthcare provider that is assessed by the Index Assessment System 100 as described in greater detail herein. Cost of care (also referred to herein as “patient spend”) is categorized or allocated into episodes of different types. For example, PCPs may be assigned an entire year of cost of care for each patient; and specialists may be either assigned to episodes of cost of care focused on acute procedures, or assigned an entire year spend for each patient with a relevant chronic condition. The Index Assessment System 100 may attribute episodes, cost of care, and/or outcomes for each of these categories based on extensions of CMS algorithms. The Index Assessment System 100 may incorporate a series of modifications of the Comprehensive Primary Care Plus (CPC+) algorithm to assign patients to primary-care providers, as CPC+ is primary care focused and is generally accepted by payers and providers across the country as a means for attribution. The Index Assessment System 100 may attribute patients to providers based on a plurality of PCP costs with a focus on services that relate to patient management, such as wellness visits and chronic condition management. For specialists managing longer-term and/or chronic conditions of patients, the Index Assessment System 100 assigns patients may use a unique collection and sequence of algorithms focusing on a plurality of patient management costs associated with each condition.

The Index Assessment System 100 may attribute acute episodes to specialists based on a modified version of the Medicare Spend per Beneficiary (MSPB) episode algorithm. The Index Assessment System 100 may assign, for example, ninety days of patient longitudinal costs after an inpatient (IP) trigger event to the provider performing the trigger procedure. The Index Assessment System 100 may extend this exemplary algorithm to trigger based upon outpatient (OP) procedures with the same ninety-day window post-procedure as the MSPB grouping algorithm. Once a cost is bundled and assigned to a relevant physician, the Index Assessment System 100 may calculate an expected value for each episode based on an ordinary least squares regression over a set of about 100 covariates, which may include patient comorbidity history and procedures. This may account for effects outside the physician's control. A doctor's overall observed-to-expected ratio for performance over the set of procedures may be calculated by the Index Assessment System 100, and a winsorization procedure may be used to limit the effect of outliers on the physician performance.

The observed-to-expected ratio may be normalized by the Index Assessment System 100 by comparing each provider to the cohort of peers in the same market, designated by the core-based statistical area. This may minimize the effects of regional variation and make the score hierarchy a comparison among a set of reasonably replaceable options. The score for each provider may be based on the test statistic of the hypothesis that the provider's observed-to-expected ratio is the same as the average observed-to-expected ratio of peers. The provider score of 1-5 produced by the Index Assessment System 100 may be the quintile of that provider's test statistic. For providers with a cost score of 5, the provider's episodes result in significantly lower costs than peers. For providers with a cost score of 1, the provider's episodes may result in significantly higher cost than peers.

Further, the Index Assessment System 100 calculates outcome scores over the same bundle. In the present example, the Index Assessment System 100 may focus on three sets of outcomes, including but not limited to, claims quality measures related to appropriateness of care for certain specialties, potentially avoidable admissions (based on the open AHRQ Prevention Quality Indicators (PQI) measures), and readmissions (based on CMS readmission algorithm). In some embodiments these measures may be tied to outcomes that are important for gauging the quality of networks of care for multiple risk-based entities. For each provider, the Index Assessment System 100 may use a weight related to size of statistics on the measures appropriate to that provider to calculate an overall measure score. The scores may be normalized across a set of peers for each provider defined by specialty and geography in the same way as the cost score. The Index Assessment System 100 may continually enrich this aspect of the score with additional open measures of quality and develop ways to tailor outcomes to customer needs.

The Index Assessment System 100 may provide a rating system that includes about 10 years of 100% of fully identifiable Medicare Fee-For-Service (FFS) claims data, which represents about 60 million lives. In other words, the Index Assessment System 100 may be configured to access the complete, linked Part A, B, and D Medicare FFS data as well as Medicare Advantage encounters data, representing over 60 million beneficiaries. Some embodiments may include claims data from non-Medicare sources. This data included in the Index Assessment System 100 is one of the largest samples of fully identifiable data for a single payer and has been validated and leveraged by academic researchers for decades. The dataset contains up-to-date claims aged 90-180 days from date of service and constitutes detailed information on diagnosis, services, dates, drugs, and providers at the patient encounter level. The Index Assessment System 100 may apply a unique combination of analytic techniques to include valid beneficiaries for study.

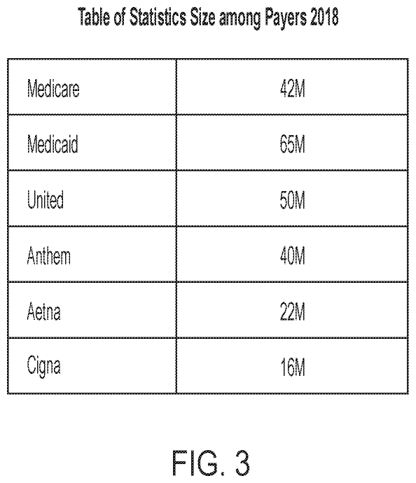

Referring now to FIG. 3, an illustrative table of statistics depicting sizes among payers in 2018 is depicted. To make statistically relevant conclusions where the levels of variation are in the 10% range, especially where there are large variations in individual patient and episode costs, significant patient samples, e.g., at least about 50 to 100, are preferred. The average provider panel in Medicare is around 200 patients, such that the entire sample may be required to provide a useful provider level metric. In some embodiments, the Index Assessment System 100 accesses the national sample of MDS and OASIS assessment data from Skilled Nursing Facilities and Home Health Agencies. Thus, the Index Assessment System 100 can provide a robust resource of patient panels at the level of activities of daily living, thereby providing more effective risk-adjustment. In other embodiments, the Index Assessment System 100 accesses the full Medicaid claims data sample, such that the full data sample considered is extended to 120 million beneficiaries, and episodes of greater relevance to a younger population are provided. In other words, the Index Assessment System 100 may be configured to access a variety of data (e.g., Medicaid data) for use by its scoring algorithms. As such, the Index Assessment 100 can achieve an analytic population of at least about 130 million. In some embodiments, the data would include 10 years of claims; and, in some embodiments, the claims would be as recent as the latest 90-180 days. The resulting scores produced by the Index Assessment System 100 may thus be highly statistically significant and timely.

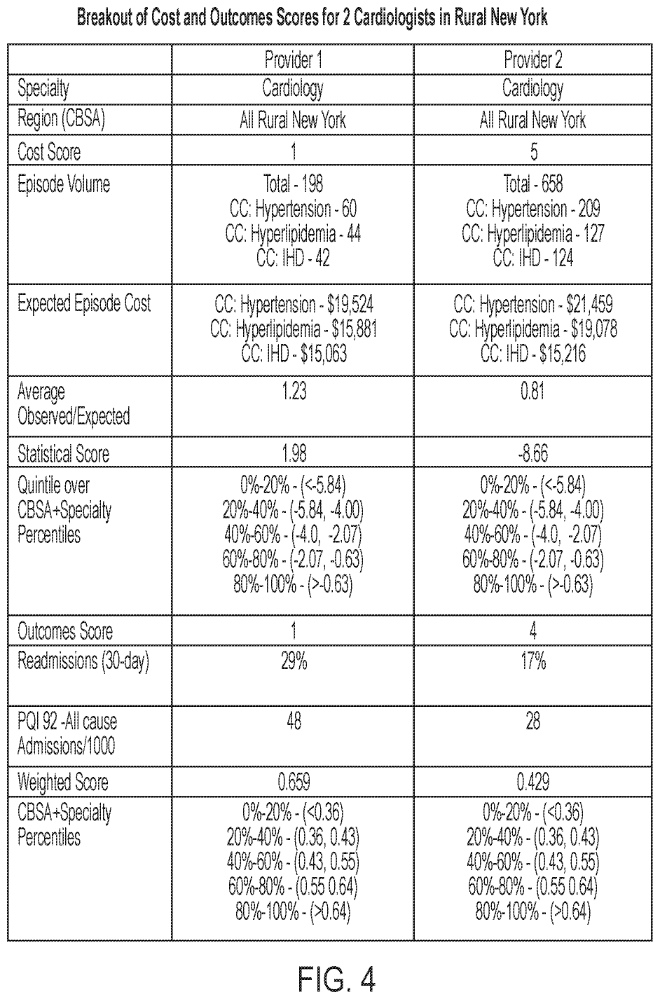

Referring now to FIG. 4, an illustrative schematic of a table including breakout costs and outcome scores for two service providers (e.g., cardiologists) in a designated location (e.g., rural New York) is depicted as an illustrative example. In this example, the Index Assessment System 100 goes through in detail the algorithm for two cardiologists who score differently in the same CBSA for the same specialty, i.e. cardiologists in rural New York State. Both may have episode volume and panel sizes that are quite large. These cardiologists may fall primarily into a category for the management of patients with chronic conditions. They may be assigned patients with chronic conditions based on a unique constellation of algorithms, and scored based on the total cost of care of these patients over the course of a year, and a standard set of outcomes over that year. Investigating the actual episodes, labeled “CC”, the Index Assessment System 100 identifies that both providers may manage a panel with similar conditions. However, the second provider's (i.e. Provider 2) expected episode costs may be higher per episode than the first provider's (i.e. Provider 1). The expected episode cost may be based on a fit, which includes co-morbidities and patient demographics. This means that the second provider's patient panel may present with a more complicated profile. Nevertheless, the actual cost for the second provider's attributed patients may be much lower compared to the actual costs for the first provider. In fact, the statistical comparison of the provider's observed to expected cost ration compared to all cardiologists suggests that the first provider's patients have much higher costs compared to his peers (z-score of 1.98) compared to the second provider (z-score −8.66). Breaking these scores into quintiles for cardiologists in rural New York, it is apparent that the second provider falls into the lowest quintile, getting a score of 5, while the first provider falls into the highest quintile, getting a score of 1.

The outcomes score may be constructed by the Index Assessment System 100 from a number of outcome measures for the provider's attributed populations. For these providers the dominant outcomes measures that are currently calculated may be the readmission rate for inpatient procedures and the AHRQ PQI-92 rate of all-cause preventable in-patient admissions. The second provider is lower on both measures and therefore the second provider's combined outcomes score is much lower than the first provider. Comparison to the percentiles for cardiologists in the region generates the scores of 1 and 4. It should be understood that the Index Assessment System 100 may allow multiple actors in the healthcare industry to compare providers using a universal scoring system that is generally built upon a few hallmark features. First, it is a compact method of consuming valuable information about a provider as the Index Assessment System 100 collapses a plurality of detailed calculations into a simplified scoring rubric that represents the doctors by at least two scores. The simplicity of the numbering schema may hide the complexity of the algorithm, in which providers with vastly different patient panels and treatments are evaluated, risk-adjusted, and compared in a coherent way. The score may be available for both PCPs and other providers.

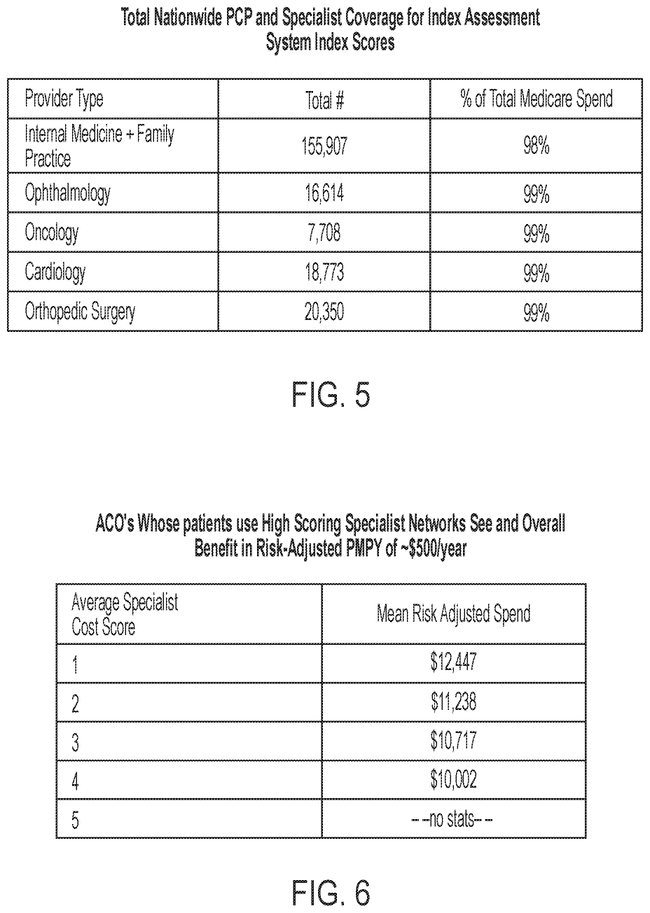

Referring now to FIG. 5, an illustrative schematic of a table including providers scored for five top specialty types is depicted, with these scored providers representing the clear majority of health care costs for their specialty. Doctors may be scored on what happens to their patients during procedures or office visits, and on the downstream costs and outcomes the patients incur afterward. This construction recognizes that the providers may be gateways to follow-up patient costs due to the decisions they make in the office, including whether or not to order an unnecessary test, how a procedure is performed, whether adequate follow-up care is coordinated, and which providers a patient is referred to downstream. For the cost score, this may de-prioritize the unit costs of provider actions and instead focus on the effects of provider decisions. The scores may correlate with observed effects in a network. Constructing a network with providers having better cost scores than other replaceable options may result in a network with measurably better costs.

Referring now to FIG. 6, an illustrative schematic of a table including overall risk-adjusted costs of care for patients in ACO's against the average specialist provider score for the network utilized by their patients is depicted. The table of FIG. 6 demonstrates that constructing networks with high scoring specialists can have measureable effects on the risk-adjusted cost of managed populations, either payers or providers in risk-based contracts. In other words, ACO's whose patients use high scoring specialist networks may receive an overall benefit in risk-adjusted costs per member per year (PMPY) of about $500 per year. The Index Assessment System 100 has the component algorithms for attribution, risk-adjustment and episode bundling to extensions of CMS algorithms. This may make the overall algorithm of the Index Assessment System 100 more auditable and comprehensive as it is based on reliable formulations.

It should be understood that for payers moving into a new market, access to provider data may be limited. The Index Assessment System 100 may provide an augmented view of provider behavior that is based on a large and orthogonal dataset, and improves the payer's overall understanding of the provider's practicing patterns, as a supplement to the provider's own data or in cases where that data does not exist. As the cost may be relative to peers within a region, the cost score can be extrapolated to non-Medicare based payer arrangements. Unit costs are not the primary measurement of the cost score. The Index Assessment System 100 may focus on the attribution of episodes to specialists and PCPs, which takes into account not just at a provider's procedures but also decisions made by the provider that may result in downstream cost or outcomes. This may be tailored to the net effect of a provider on a network of care based on the provider's behavior and not the rates a provider charges. For this reason, the Index Assessment System 100 can extrapolate a score outside of the Medicare data. For instance, a cardiology group may consistently order unnecessary stress tests. These tests would show up as a lower score for additional utilizations, but the net effect may be more extreme since the unnecessary tests may result in unnecessary procedures that are far more costly than the test itself.

Health systems taking on risk have a number of the same may use cases as the payer above if they are taking on financial risk for managing a population at risk. The Index Assessment System 100 affiliates gain-sharing arrangement for specialist networks with an ACO or other risk-based entity. The Index Assessment System 100 may be configured to analyze the net effect of specialists on the ability of an organization to reach its cost and quality goals. An ACO could use the specialist cost or quality rating as a weighting measure, along with network volume, to stratify the gain-sharing across a specialist network. Physicians may use the Index Assessment System 100 to audit and understand their own performance. Many physicians may not understand the downstream effects of their decisions.

The Index Assessment System 100 may provide a longitudinal view of patient outcomes and costs after patients leave the provider's office, and how those outcomes and costs compare to the provider's peers. In some embodiments, physicians can drill-down and understand Care Models that they can institute to improve their index scores. For instance, a cardiologist may practice in a generally cost-efficient way, but be imbedded in a system that does not coordinate discharge follow-ups well. The cardiologist might receive a low score from the Index Assessment System 100 of the present disclosure as compared to peers with better coordination across the system. This is important information for the physician as payers will want to view the holistic picture of what will happen to a patient interacting with this physician, not just what happens in the physician's office. The physician could use information from the Index Assessment System 100 to drill down and discover that her transitions of care compliance were low and readmissions were high, and focus effort on coordinating better with the system.

The Index Assessment System 100 may include a claims-based algorithm. Generally, claims-based datasets may include biases and incentives that distort the representation of patient-care in claims. However, it is a comprehensive and complete source that is managed under a single data structure, and is available for every patient over their duration in Medicare. The data also may not allow access to potential confounding variables outside of the dataset, which might unduly influence the measurements made. To minimize exposure to these biases, the Index Assessment System 100 may be configured to perform risk-adjusting using algorithms and approaches.

The Index Assessment System 100 can be an evolving framework for evaluating physicians that includes one or more components configured to continuously update and improve the algorithms stored therein as new data and member feedback is received by the Index Assessment System 100. For example, the Index Assessment System 100 is configured to incorporate and develop new, collaborative methods for bundling, risk adjustment, and attribution. The Index Assessment System 100 may be configured to expand the underlying metrics in the outcomes score to build-out specialist-specific measures of quality. Since outcomes may be combined over multiple measures, the Index Assessment System 100 may be configured to analyze member specific weighting schemas to tailor the score to measures of interest. The Index Assessment System 100 may be configured to enable physicians to audit their performance, and to focus their assessment on factors affecting scores, such as underlying metrics, underlying risk of the provider's patient mix, the provider's referral network, and the provider's procedure volume. The Index Assessment System 100 may generate relevant care models that the provider can institute to improve the outcomes or costs for the patient panel, including relevant patient segments, on which to focus effort to enhance care and improve scores. The Index Assessment System 100 may be configured such that it is a useful tool for evaluating healthcare provider behavior, and it facilitates a generated plan, report, and/or representation to improve healthcare generally.

The Index Assessment System 100 may also be used to continuously improve scoring and recommendations generated therefrom as a result of the Index Assessment System 100 continually accessing new data sources. Social determinants of health data and data related to activities of daily living, obtainable through MDS and OASIS survey data as well as other open sources can improve risk adjustments and the impact of recommendations for provider improvement generated by the Index Assessment System 100. Commercial and Medicaid claims can improve the statistical power of conclusions, and will open new opportunities for improved health care with greater cost-effectiveness.

The Index Assessment System 100 may be configured to provide a variety of benefits, including, for example: being usable to a non-technical consumer; factoring in patient outcomes over a longer timeline a single procedure or stay (i.e. based on the net effects of provider decisions on the course of a patient's care); factoring-out biases and artifacts that arise from not adequately applying appropriate statistical treatments or risk adjustment methodologies; and ratings tied to actual patient outcomes and/or cost that a consumer may expect to hold. Additionally, the Index Assessment System 100 may provide: a rating that does not migrate arbitrarily over-time unless the underlying performance changes, is based on open-methodologies, uses an overall rating that is tied to fundamental factors that a healthcare provider can analyze in detail to improve services and/or outcomes, continuous improvement as new requirements are produced, and is modifiable for different use-cases. Further, the Index Assessment System 100 may provide actions that the provider can focus and improve upon when generating a poor provider rating, and the rating system exist for PCPs and specialists who manage both acute and chronic conditions.

It should now be understood that the Index Assessment System 100 may be used in various ways and allows multiple actors in the healthcare industry to assess performance and/or compare providers using a universal scoring system. For example, third-party payers may use it to evaluate physicians for inclusion in the networks for their plans. The rating produced by the Index Assessment System 100 may simplify decisions among replaceable peers in a region, and makes predictions about how a decision point might affect a key performance indicator for that network. For pre-existing networks, payers may want to identify high or low performances for special contracting arrangements, incentive programs, or for replacement opportunities. Through the Index Assessment System 100, payers can receive an augmented view of provider behavior that is based on a large and orthogonal dataset to improve overall understanding of a provider's performance and practicing patterns, as a supplement to the payer's own data, or where that data does not exist. The Index Assessment System 100 may allow payers to evaluate providers for inclusion/exclusion in a network or make predictions about how a decision might affect a key performance indicator for that network. For pre-existing networks, the Index Assessment System 100 allows payers to identify high or low performers for special contracting arrangements, incentive programs, or for replacement opportunities.

Additionally, the Index Assessment System may be used by health systems that take on risk who need to create a network of care tied to risk-based contracts that put them in position for success. For successful risk-based arrangements, they put-in-place gain-sharing agreements, which can be especially difficult for specialist networks. By providing a rating that is public-facing via the Index Assessment System 100, health systems may need to manage and maintain their cost and quality standards by acquiring high-scoring providers and incenting behavior change in low-scoring providers. Through the Index Assessment System 100, health systems can better assess the net effect of specialists on their ability to reach cost and outcome goals. Health systems may use the Specialist cost or outcome rating as a weighting measure, along with network volume, to stratify the gain-sharing across a Specialist network.

Furthermore, physicians generally have access to their own data but may not have a full picture of what happens to their patients after they leave the office. A provider score produced by the Index Assessment System 100 can show physicians how they compare to peers in their region, and detailed analysis of the data offers them the ability to take action on efficiency or quality deficiencies. In other words, the Index Assessment System 100 may allow physicians to audit themselves, and better understand their own performance against peers; and, to take action on efficiency or quality deficiencies. In some embodiments, physicians will be able to drill-down and understand Care Models they can institute to improve care, and corresponding index scores. Lastly, patients may be continually presented with choices in their own care, especially related to which provider might manage their procedure or condition. Data to inform their decision may be provided by the Index Assessment System 100 through a concise, universal provider rating system tied to downstream outcomes and cost.

The Index Assessment System 100 can normalize scores and compare providers that treat similar populations, while also taking an episodic approach based on the MSPB metric, which can be meaningful for specialists.

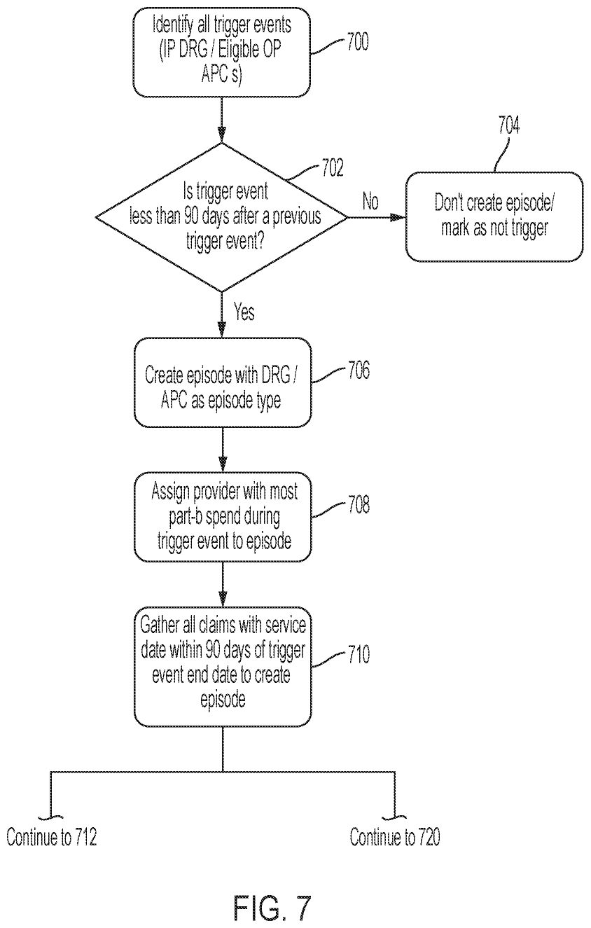

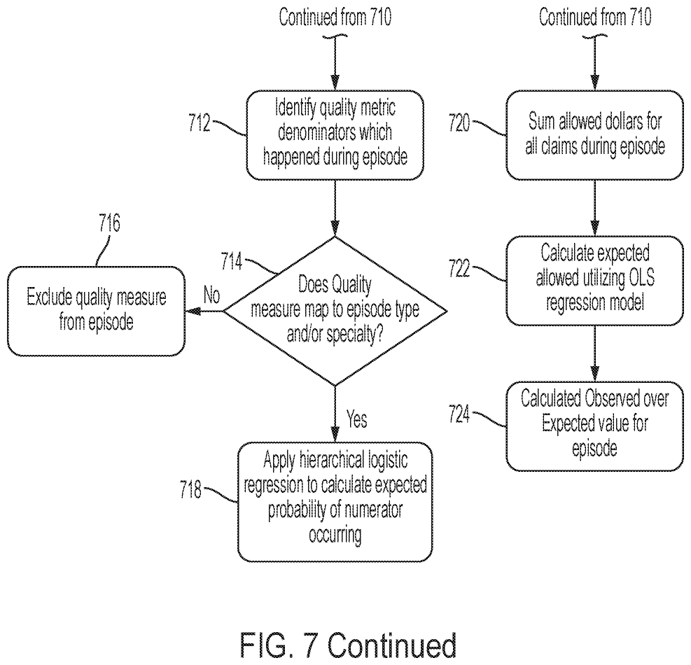

Referring now to FIG. 7, a flow chart depicting acute episodes according to one or more embodiments shown and described herein. At block 700, all potential trigger events that are eligible for inpatient and outpatient claims may be identified. For inpatient claims, for example, any DRG may be treated as a potential trigger event. For outpatient claims, any outpatient procedure code in the Healthcare Common Procedure Coding (HCPC) system that makes up at least 2% of a specialty spend may be picked. Those HCPC's may be mapped up to an Ambulatory Payment Classification (APC) level. APCs are essentially the outpatient equivalent of a DRG, and any claim with an APC identified this way may be considered an eligible trigger event for the given specialty. Trigger event can last as short as a day, or many days. In order to assign the physician responsible, all part b claims that happened on days during the trigger event are taken, and such that allowed dollars by physician may be summed. For example, the physician who had the highest spend gets assigned the episode.

At block 704, a determination is made as to whether a trigger event is less than a threshold amount of time, such as 90 days, after a previous trigger event. In this embodiment, all potential trigger events for a beneficiary in a given claim type are taken, and any events that happened can be removed (inpatient and outpatient are done separately in this embodiment). By way of non-limiting example, a beneficiary has 4 inpatient claims in a calendar year as follows: (i) Jan. 10, 2019—Trigger event, gets 90-day episode; (ii) Feb. 10, 2019—not a trigger event since it is within 90 days of previous trigger, and thus no episode; Apr. 25, 2019—trigger event, more than 90 days from previous trigger (i), and thus gets an episode; Jun. 10, 2019—not a trigger event since it is within 90 days of previous trigger, and thus there is no episode.

If there is no trigger event within the threshold amount of time after a previous trigger event, then at block 704, there is no episode created and/or it is not marked as trigger. Otherwise, if there is a trigger event within the threshold amount of time after a previous trigger event, then at block 706, an episode with a DRG/APC type is created.

At block 708, once the episodes are created, an episode type is assigned to an episode. The episode type may be either the DRG or APC code of the trigger event, for example. Episode types may include primary care, chronic conditions, Medicare Spending per Beneficiary (MSPB) acute inpatient/outpatient, and the like. Primary care episodes can relate to patient care management for primary care physicians, and provide consideration for provider treatment of patients by frailty cohort. Chronic condition episodes may relate to chronic condition management for specialist physicians and may be calculated for the most “acute” chronic condition a patient has (identified as the chronic condition with the highest average episode spend across all patients). In some embodiments, for a provider to be assigned a chronic condition episode, they need to be eligible to treat that chronic condition (e.g., orthopedic surgeons can't be assigned heart failure episodes).

Acute inpatient episodes may start with a trigger event 90-day cost of care logic post-discharge (or any other suitable duration) and may correspond to a specific DRG from the trigger event. Acute outpatient episodes may start with a trigger event (which may be clearly defined in claims, even without an inpatient stay) and utilize a 90-day cost of care logic post-discharge (or any other suitable duration). Acute outpatient episodes may correspond to a specific Ambulatory Payment Classification (APC) from the trigger event (APC is the outpatient equivalent of a DRG). In this embodiment, providers must be “eligible” to treat specific acute episodes.

At block 710, all claims that happened either during the trigger event, or within 90 days of the end of the trigger event (Parts A and B) may be gathered. These claims may be identified, for example, by the service date for part B, and the discharge date for part A. A quality score and a cost score may then be identified, as described further below. Block 710 proceeds to both blocks 712 and 720.

At block 712, it is identified whether quality measures can be linked to episodes, based upon quality metric denominators that happened during an episode. Using a binomial distribution allows consideration of providers with no metric occurrences. A binominal distribution model is used to hold each provider accountable to their own performance and patient population. It allows, for each provider, a determination as to whether the probability of performing as well or better than they actually did, based on what would be expected given the demographics and health factors of their patient population. The avoidable Emergency Department (ED) rate is provided by formula 1 as follows:

$$P ( x ) ( \begin{matrix}n \\ x\end{matrix} ) {p}^{x} {q}^{n - x} = \frac{n !}{( n - x ) ! x !} {p}^{x} {q}^{n - x} \qquad Formula 1$$

Numerator contribution: # of ED visits that were avoidable

Denominator contribution: All ED visits

n=# of total observations (denominator)

x=# of metric occurrences (numerator)

p=expected metric outcome probability

q=1-p, probability of not seeing metric outcome

In this embodiment, there are two types of quality measures denominators. First are claims based which look at the date of the claim where an episode can have more than 1 denominator if they have multiple claims. An example measure includes avoidable ED visits, if a beneficiary has four ED visits, each may be treated as a denominator event and predict the probability that each is an avoidable visit. The second type of quality measures are episode-based, which are driven by the beneficiary, and each episode will have only one denominator in this embodiment. An example measure is in terms of PQI's, wherein each episode has a denominator of 1, with a probability prediction of that episode having at least one PQI.

At block 714, a determination is made as to whether a quality measure maps to an episode type and/or specialty. Put another way, it is determined whether the measure is appropriate for the episode type/specialty based on an internal mapping. If the quality measure maps to neither an episode type nor a specialty, then at block 716 the quality measure may be excluded from the episode. Otherwise, if there is a mapping, then at block 718 a hierarchical logistic regression may be used to calculate an expected probability of a numerator occurring. Specifically, a hierarchical logistic regression may be applied with the episode type being a random intercept to determine the probability of the numerator being met for each denominator.

At block 720, once all the claims have been determined, the total allowed dollars may be summed to get a total allowed per episode. At block 722, an expected allowed amount may be calculated utilizing Ordinary Least Squares (OLS) regression model for each Major Diagnostic Category (MDC) for inpatient episodes and specialty for outpatient episodes. At block 724, the calculated observed is divided by the expected value for the episode.

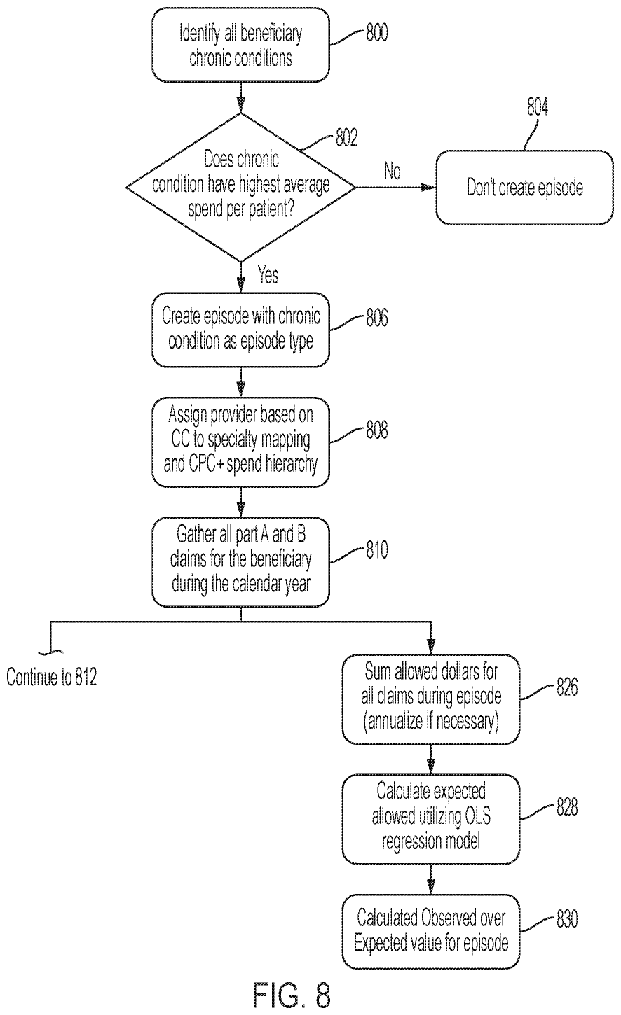

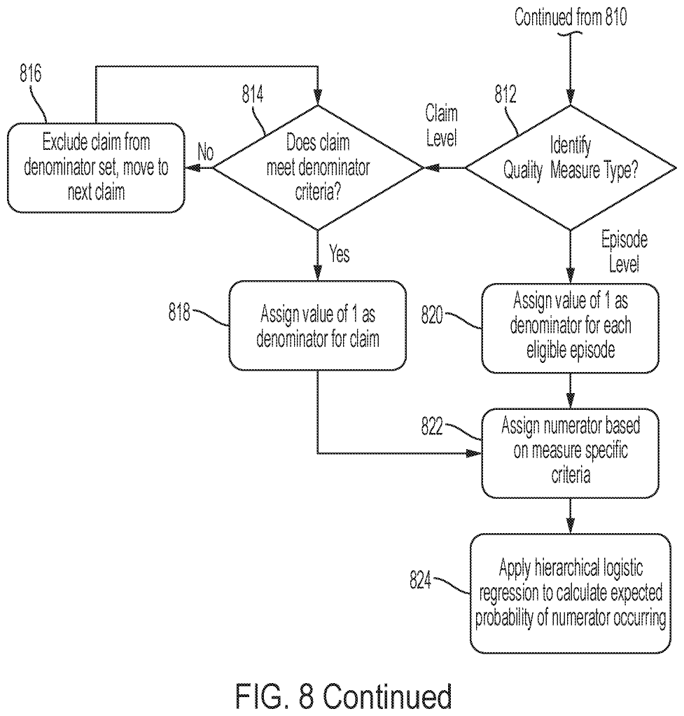

Referring now to FIG. 8, a flow chart depicting chronic condition episodes according to one or more embodiments shown and described herein. At block 800 some or all potential chronic conditions that a beneficiary has based on mapping the CMS chronic conditions.

At block 802 a determination is made as to whether a chronic condition has the highest average spend per patient. Each chronic condition may be mapped to an internal hierarchy, and only the chronic condition that is the highest on the list will get assigned as an episode for that beneficiary. In other words, the most expensive chronic condition on average that a beneficiary has may be turned into an episode, and the other chronic conditions may not become episodes.

For example, a beneficiary has three chronic conditions: lung cancer, hypertension, and osteoporosis, with associated costs as follows:

Average cost of beneficiary with lung cancer: $200,000—assigned an episode.

Average cost of beneficiary with hypertension: $60,000—not assigned an episode.

Average cost of beneficiary with osteoporosis: $140,000—not assigned an episode.

If, at block 804, a chronic condition does not have a highest average cost among chronic conditions affecting a beneficiary, then it is not assigned an episode. Conversely, if a chronic condition does have a highest average cost among chronic conditions affecting a beneficiary, then at block 806 an episode may be created with the chronic condition as an episode type.

At block 808 a provider may be assigned based on a mapping of the chronic condition to the specialty and CPC+ spend hierarchy. To attribute chronic condition and primary care episodes, a modified version of CPC+ attribution logic may consider claims in a hierarchy, starting with Chronic Condition Management (CCM) claims, then Annual Wellness Visit (AWV) claims, and then all other Evaluation & Management (E&M) claims. In one embodiment, patients may be attributed to the specialist in an “eligible” specialty billing the highest allowed E&M dollars during the year by chronic condition episode type. E&M dollars may then be evaluated in a hierarchy that is in line with the CMS CPC+ methodology, where CCM dollars are evaluated first, AWV dollars are evaluated second and all other E&M dollars are evaluated last if there are no CCM or AWV dollars.

At block 810, all claims (Parts A and B) that happened during the calendar year (or any other suitable timeframe) may be gathered. The claims may relate by service date for part B, and discharge date for part A. From block 810, the flowchart branches to block 812 and block 826. At block 812 the quality measure type is identified if it is a claim level or an episode level. If it is a claim level, then at block 814 a determination may be made as to whether the claim meets the denominator criteria. Claims which look at the date of the claim where an episode can have more than one denominator if they have multiple claims. For example, in the context of avoidable emergency department (ED) visits, if a patient has four ED visits, each may be treated as a denominator event with a prediction of the probability that each is an avoidable visit.

If the claim does not meet the denominator criteria at block 814, then at block 816 the claim may be excluded from the denominator set and the next claim may be considered. If, however, the claim meets denominator criteria, then at block 814 the flowchart proceeds to block 818 where a value of one (or other appropriate value in other embodiments) may be assigned to the claim, and the flowchart proceeds to block 822. Returning to block 812, if the quality measure type is identified is an episode level, then at block 820 a value of one (or other appropriate value in other embodiments) may be assigned to the claim. As an example measure, in the context of PQI's, each episode may have a denominator of 1, such that the probability of that episode may be predicted as having at least 1 PQI. At block 822 the numerator may be assigned based upon measure-specific criteria. At block 824 apply a hierarchical logistic regression with the episode type being a random intercept to determine the probability of the numerator being met for each denominator

At block 826, once all the claims have been determined, the total allowed dollars may be summed to get a total allowed per episode. At block 828, an expected allowed amount may be calculated utilizing OLS regression model for each MDC for inpatient episodes and specialty for outpatient episodes. At block 830, the calculated observed may be divided by the expected value for the episode.

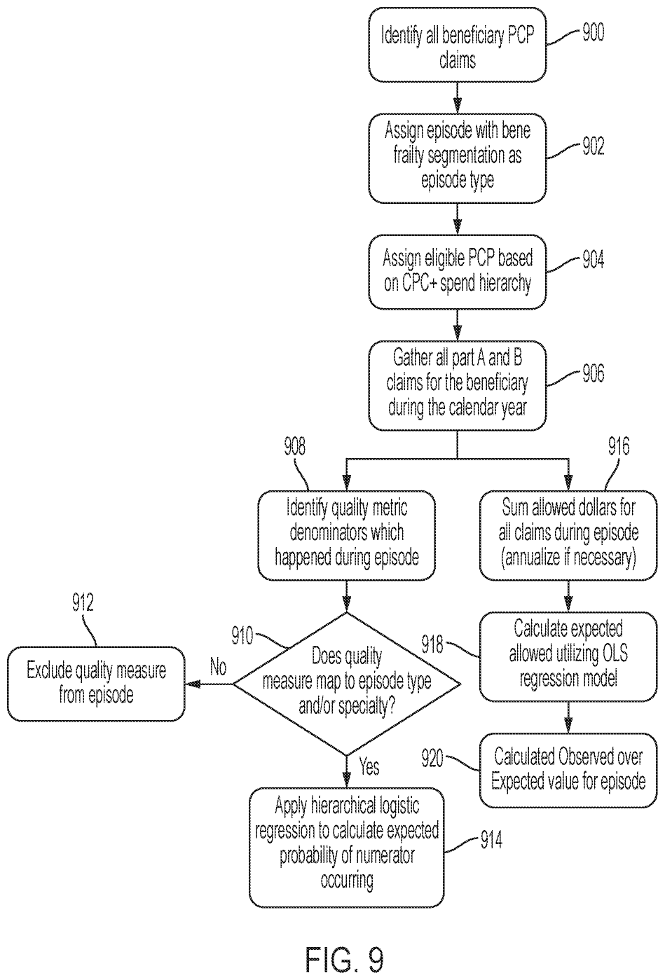

Referring now to FIG. 9, a flowchart depicting primary care physician (PCP) episodes according to one or more embodiments shown and described herein. At block 900 all PCP claims that a patient has are identified. At block 902 an episode may be assigned to each patient with a PCP claim, where the episode type is their frailty segmentation. At block 904 a provider may be assigned based on a mapping of the chronic condition to the specialty and/or CPC+ spend hierarchy. To attribute chronic condition and primary care episodes, a modified version of CPC+ attribution logic may consider claims in a hierarchy, starting with Chronic Condition Management (CCM) claims, then Annual Wellness Visit (AWV) claims, and then all other Evaluation & Management (E&M) claims. In one embodiment, patients may be attributed to the specialist in an “eligible” specialty billing the highest allowed E&M dollars during the year by chronic condition episode type. E&M dollars may then be evaluated in a hierarchy that is in line with the CMS CPC+ methodology, where CCM dollars are evaluated first, AWV dollars are evaluated second and all other E&M dollars are evaluated last if there are no CCM or AWV dollars.

At block 906 all claims (Parts A and B) that happened during the calendar year (or any other suitable timeframe) may be gathered. The claims may relate by service date for part B, and discharge date for part A. At block 908 it is identified whether quality measures can be linked to episodes, based upon quality metric denominators that happened during an episode. Using a binomial distribution allows consideration of providers with no metric occurrences. A binominal distribution model is used to hold each provider accountable to their own performance and patient population. It allows, for each provider, a determination as to whether the probability of performing as well or better than they actually did, based on what would be expected given the demographics and health factors of their patient population.

At block 910 a determination is made as to whether a quality measure maps to an episode type and/or a specialty. If not, then at block 912 the quality measure is excluded from the episode. Otherwise, if there is a mapping, then at block 914 a hierarchical logistic regression may be used to calculate an expected probability of a numerator occurring. Specifically, a hierarchical logistic regression may be applied with the episode type being a random intercept to determine the probability of the numerator being met for each denominator.

At block 916, once all the claims have been determined, the total allowed dollars may be summed to get a total allowed per episode. At block 918, an expected allowed amount may be calculated utilizing an OLS regression model for each MDC for inpatient episodes and specialty for outpatient episodes. At block 920, the calculated observed cost (or value) may be divided by the expected cost (or value) for the episode.

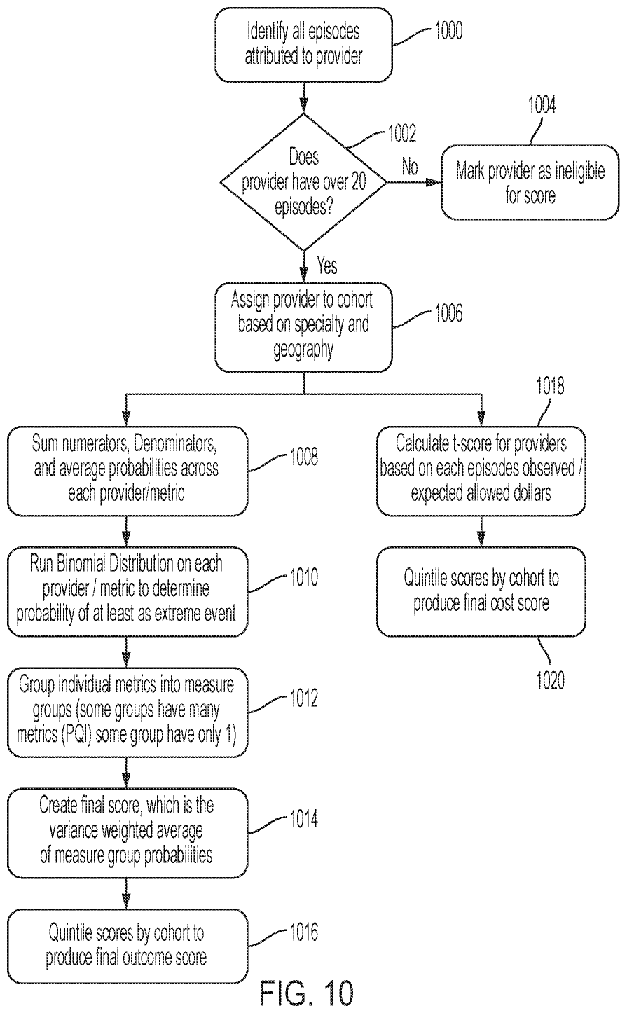

Referring now to FIG. 10, a flowchart depicting primary care physician (PCP) episodes according to one or more embodiments shown and described herein. At block 1000 all episodes attributed to a provider may be identified. This may include, for example, some or all episodes created via acute episodes, chronic episodes, PCP episodes, and the like. At block 1002, a determination is made as to whether the provider exceeds a threshold number of episodes, such as 20 episodes in this non-limiting example. If not, then at block 1004 the provider is deemed ineligible for a score because there may not be a statistically sound rating for that doctor, and therefore the provider may be marked as ineligible for rating. If the provider has more than 20 episodes (or any other suitable threshold), however, then at block 1006 the provider may be assigned to a cohort based on their specialty and/or geography. The flowchart then branches from block 1006 to block 1008 and block 1018.

At block 1008, an aggregate summary of each metric for each provider may be obtained by summing the numerators and denominators calculated in the episode portion, and averaging the probabilities from the logistic regression for each measure. At block 1010, a binomial distribution may be run on each provider/metric from 1008 to obtain a probability of a provider performing at least as well as what was observed. In this embodiment, the binomial may take three arguments: (i) the number of trials=sum of denominators; (ii) the number of events=sum of numerators; and (iii) probability of event=average logistic probability.

At block 1012, metrics may be placed into similar groups. By way of non-limiting example, AHRQ PQI's are a first group and AHRQ PSI's are a second group, wherein every other measure is its own group. An average probability may then be calculated based upon the data from block 1010. At block 1014, a final score may be created, which in this embodiment is the variance weighted average probabilities of the groups. The measures may be weighted by variance to confer greater benefit upon a provider doing well on a metric where performance is evenly spread, rather than for a metric where all (or most) providers do well. At block 1016, t-scores of the final scores may be quintiled into 5 groups, assigning a 1 to the worst performing group and a 5 to the best performing based for each cohort from block 1006 to obtain a final outcomes score for each provider and a final quality index. Any suitable rating scale and/or number of groups may be utilized in other embodiments.

At block 1018, an aggregated t-score may be calculated to determine how much the result varies from the average or mean based on each provider's observed/expected values for the episodes to which they are attributed. At block 1020, the t-scores may be quintiled into 5 groups, for example assigning a 1 to the worst performing group and a 5 to the best performing based for each cohort from block 1012 to obtain a final cost index. Any suitable rating scale and/or number of groups may be utilized in other embodiments.

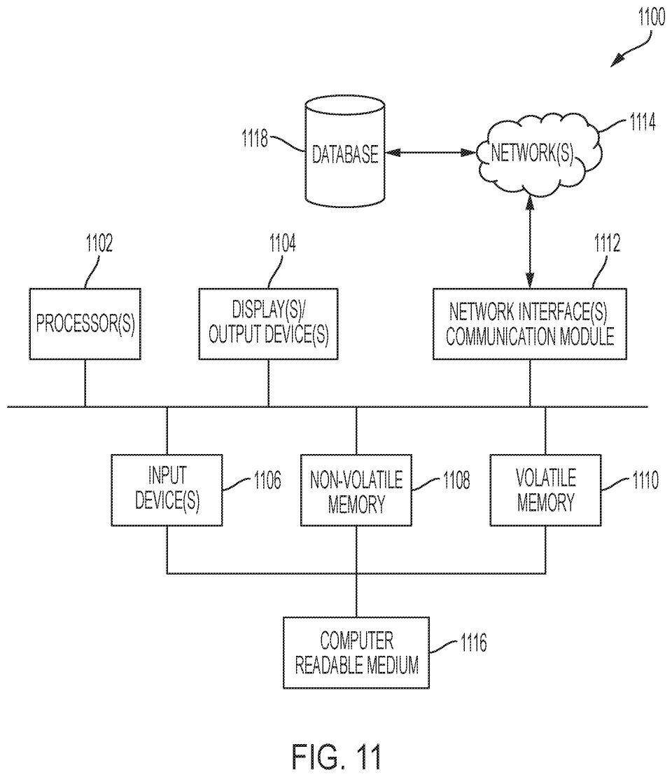

Referring now to FIG. 11, a block diagram illustrates an exemplary computing device 1100, through which embodiments of the disclosure can be implemented. The computing device 1100 described herein is but one example of a suitable computing device and does not suggest any limitation on the scope of any embodiments presented. Nothing illustrated or described with respect to the computing device 1100 should be interpreted as being required or as creating any type of dependency with respect to any element or plurality of elements. In various embodiments, a computing device 1100 may include, but need not be limited to, a desktop, laptop, server, client, tablet, smartphone, or any other type of device that can compress data. In an embodiment, the computing device 1100 includes at least one processor 1102 and memory (non-volatile memory 1108 and/or volatile memory 1110). The computing device 1100 can include one or more displays and/or output devices 1104 such as monitors, speakers, headphones, projectors, wearable-displays, holographic displays, and/or printers, for example. Output devices 1104 may further include, for example, audio speakers, devices that emit energy (radio, microwave, infrared, visible light, ultraviolet, x-ray and gamma ray), electronic output devices (Wi-Fi, radar, laser, etc.), audio (of any frequency), etc.

The computing device 1100 may further include one or more input devices 1106 which can include, by way of example, any type of mouse, keyboard, disk/media drive, memory stick/thumb-drive, memory card, pen, touch-input device, biometric scanner, voice/auditory input device, motion-detector, camera, scale, and the like. Input devices 1106 may further include sensors, such as biometric (blood pressure, pulse, heart rate, perspiration, temperature, voice, facial-recognition, iris or other types of eye recognition, hand geometry, fingerprint, DNA, dental records, weight, or any other suitable type of biometric data, etc.), video/still images, motion data (accelerometer, GPS, magnetometer, gyroscope, etc.) and audio (including ultrasonic sound waves). Input devices 1106 may further include cameras (with or without audio recording), such as digital and/or analog cameras, still cameras, video cameras, thermal imaging cameras, infrared cameras, cameras with a charge-couple display, night-vision cameras, three-dimensional cameras, webcams, audio recorders, and the like.

The computing device 1100 typically includes non-volatile memory 1108 (ROM, flash memory, etc.), volatile memory 1110 (RAM, etc.), or a combination thereof. A network interface 1112 can facilitate communications over a network 1114 via wires, via a wide area network, via a local area network, via a personal area network, via a cellular network, via a satellite network, etc. Suitable local area networks may include wired Ethernet and/or wireless technologies such as, for example, wireless fidelity (Wi-Fi). Suitable personal area networks may include wireless technologies such as, for example, IrDA, Bluetooth, Wireless USB, Z-Wave, ZigBee, and/or other near field communication protocols. Suitable personal area networks may similarly include wired computer buses such as, for example, USB and FireWire. Suitable cellular networks include, but are not limited to, technologies such as LTE, WiMAX, UMTS, CDMA, and GSM. Network interface 1112 can be communicatively coupled to any device capable of transmitting and/or receiving data via the network 1114. Accordingly, the network interface hardware 1112 can include a communication transceiver for sending and/or receiving any wired or wireless communication. For example, the network interface hardware 1112 may include an antenna, a modem, LAN port, Wi-Fi card, WiMax card, mobile communications hardware, near-field communication hardware, satellite communication hardware and/or any wired or wireless hardware for communicating with other networks and/or devices.

A computer-readable medium 1116 may comprise a plurality of computer readable mediums, each of which may be either a computer readable storage medium or a computer readable signal medium. A computer readable storage medium 1116 may reside, for example, within an input device 1106, non-volatile memory 1108, volatile memory 1110, or any combination thereof. A computer readable storage medium can include tangible media that is able to store instructions associated with, or used by, a device or system. A computer readable storage medium includes, by way of example: RAM, ROM, cache, fiber optics, EPROM/Flash memory, CD/DVD/BD-ROM, hard disk drives, solid-state storage, optical or magnetic storage devices, diskettes, electrical connections having a wire, or any combination thereof. A computer readable storage medium may also include, for example, a system or device that is of a magnetic, optical, semiconductor, or electronic type. Computer readable storage media and computer readable signal media are mutually exclusive.

A computer readable signal medium can include any type of computer readable medium that is not a computer readable storage medium and may include, for example, propagated signals taking any number of forms such as optical, electromagnetic, or a combination thereof. A computer readable signal medium may include propagated data signals containing computer readable code, for example, within a carrier wave. Computer readable storage media and computer readable signal media are mutually exclusive.

The computing device 1100 may include one or more network interfaces 1112 to facilitate communication with one or more remote devices, which may include, for example, client and/or server devices. A network interface 1112 may also be described as a communications module, as these terms may be used interchangeably. A database 1118 may be remotely accessible on a server or other distributed device and/or stored locally in the computing device 1100.

It is noted that recitations herein of a component of the present disclosure being “configured” or “programmed” in a particular way, to embody a particular property, or to function in a particular manner, are structural recitations, as opposed to recitations of intended use. More specifically, the references herein to the manner in which a component is “configured” or “programmed” denotes an existing physical condition of the component and, as such, is to be taken as a definite recitation of the structural characteristics of the component.

The order of execution or performance of the operations in examples of the disclosure illustrated and described herein is not essential, unless otherwise specified. That is, the operations may be performed in any order, unless otherwise specified, and examples of the disclosure may include additional or fewer operations than those disclosed herein. For example, it is contemplated that executing or performing a particular operation before, contemporaneously with, or after another operation is within the scope of aspects of the disclosure.

It is noted that the terms “substantially” and “about” and “approximately” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method comprising:
identifying potential chronic conditions a patient has based on a chronic conditions mapping;
determining a cost score;
determining an outcome score;
obtaining a final outcome score for each of a plurality of providers based on a variance weighted average of probabilities for groups of metrics pertaining to each provider; and
generating a final quality index based upon the final outcome score for each provider.

2. The method of claim 1 wherein the cost score comprises:
identifying a plurality of episodes;
attributing episodes to providers using allowed amounts;
calculating a total spend amount for each episode;
determining an expected cost of each episode;
calculating a composite score for each provider; and
calculating a final cost index score.

3. The method of claim 1, wherein the outcome score comprises:
determining attributed episodes and a patient population for each provider;
identifying a plurality of outcome metrics for the attributed episodes and the patient population;
calculating an expected probability for each outcome metric for each episode;
aggregating each outcome metric for each provider;
calculating a binomial probability of performing as well or better than the provider actually performed on each metric; and
combining outcome metrics into one score.

4. The method of claim 3 wherein the binomial probability is P(x) as defined by
$$P ( x ) ( \begin{matrix}n \\ x\end{matrix} ) {p}^{x} {q}^{n - x} = \frac{n !}{( n - x ) ! x !} {p}^{x} {q}^{n - x}$$
where n is a quantity of total observations, x is a quantity metric occurrences, p is an expected metric outcome probability, and q=1−p, where q is a probability of not seeing a metric outcome, numerator n! is a quantity of Emergency Department (ED) visits that were avoidable, and denominator (n−x)!x! is a quantity of total ED visits.

5. The method of claim 1 further comprising utilizing a hierarchical logistic regression model that takes patient risk factors into account to factor into an expected episode cost based upon a ratio of observed cost over expected cost.

6. The method of claim 5, wherein different hierarchical logistic regression models are utilized for each of primary care episodes, chronic condition episodes, acute inpatient episodes, and acute outpatient episodes.

7. The method of claim 1 further comprising mapping each chronic condition associated with a patient to an internal hierarchy wherein only a chronic condition with a highest average cost among all chronic conditions associated with the patient becomes an episode.

8. A system comprising:
memory and a processor coupled to the memory, wherein the processor is configured to:
identify potential chronic conditions a patient has based on a chronic conditions mapping;
determine a cost score;
determine an outcome score;
obtain a final outcome score for each of a plurality of providers based on a variance weighted average of probabilities for groups of metrics pertaining to each provider; and
generate a final quality index based upon the final outcome score for each provider.

9. The system of claim 8 wherein the processor is further configured to determine the outcome score by:
determining attributed episodes and a patient population for each provider;
identifying a plurality of outcome metrics for the attributed episodes and the patient population;
calculating an expected probability for each outcome metric for each episode;
aggregating each outcome metric for each provider;
calculating a binomial probability of performing as well or better than the provider actually performed on each metric; and
combining outcome metrics into one score.

10. The system of claim 8 wherein the processor is further configured to determine the outcome score by:
determining attributed episodes and a patient population for each provider;
identifying a plurality of outcome metrics for the attributed episodes and the patient population;
calculating an expected probability for each outcome metric for each episode;
aggregating each outcome metric for each provider;
calculating a binomial probability of performing as well or better than the provider actually performed on each metric; and
combining outcome metrics into one score.

11. The system of claim 10, wherein the binomial probability is P(x) as defined by
$$P ( x ) ( \begin{matrix}n \\ x\end{matrix} ) {p}^{x} {q}^{n - x} = \frac{n !}{( n - x ) ! x !} {p}^{x} {q}^{n - x}$$
where n is a quantity of total observations, x is a quantity metric occurrences, p is an expected metric outcome probability, and q=1−p, where q is a probability of not seeing a metric outcome, numerator n! is a quantity of Emergency Department (ED) visits that were avoidable, and denominator (n−x)!x! is a quantity of total ED visits.

12. The system of claim 8, wherein the processor is further configured to utilize a hierarchical logistic regression model that takes patient risk factors into account to factor into an expected episode cost based upon a ratio of observed cost over expected cost.

13. The system of claim 12, wherein different hierarchical logistic regression models are utilized for each of primary care episodes, chronic condition episodes, acute inpatient episodes, and acute outpatient episodes.

14. The system of claim 8, wherein the processor is further configured to map each chronic condition associated with a patient to an internal hierarchy, wherein only a chronic condition with a highest average cost among all chronic conditions associated with the patient becomes an episode.

15. A non-transitory computer readable medium embodying computer-executable instructions, that when executed by a processor, cause the processor to execute operations comprising:
identifying potential chronic conditions a patient has based on a chronic conditions mapping;
determining a cost score;
determining an outcome score;
obtaining a final outcome score for each of a plurality of providers based on a variance weighted average of probabilities for groups of metrics pertaining to each provider; and
generating a final quality index based upon the final outcome score for each provider.

16. The non-transitory computer readable medium of claim 15 embodying further computer-executable instructions wherein the cost score comprises:
identifying a plurality of episodes;
attributing episodes to providers using allowed amounts;
calculating a total spend amount for each episode;
determining an expected cost of each episode;
calculating a composite score for each provider; and
calculating a final cost index score.

17. The non-transitory computer readable medium of claim 15 embodying further computer-executable instructions wherein the outcome score comprises:
determining attributed episodes and a patient population for each provider;
identifying a plurality of outcome metrics for the attributed episodes and the patient population;
calculating an expected probability for each outcome metric for each episode;
aggregating each outcome metric for each provider;
calculating a binomial probability of performing as well or better than the provider actually performed on each metric; and
combining outcome metrics into one score.

18. The non-transitory computer readable medium of claim 17 embodying further computer-executable instructions wherein the binomial probability is P(x) as defined by
$$P ( x ) ( \begin{matrix}n \\ x\end{matrix} ) {p}^{x} {q}^{n - x} = \frac{n !}{( n - x ) ! x !} {p}^{x} {q}^{n - x}$$
where n is a quantity of total observations, x is a quantity metric occurrences, p is an expected metric outcome probability, and q=1−p, where q is a probability of not seeing a metric outcome, numerator n! is a quantity of Emergency Department (ED) visits that were avoidable, and denominator (n−x)!x! is a quantity of total ED visits.

19. The non-transitory computer readable medium of claim 15 embodying further computer-executable instructions that comprise utilizing a hierarchical logistic regression model that takes patient risk factors into account to factor into an expected episode cost based upon a ratio of observed cost over expected cost.

20. The non-transitory computer readable medium of claim 19 embodying further computer-executable instructions such that different hierarchical logistic regression models are utilized for each of primary care episodes, chronic condition episodes, acute inpatient episodes, and acute outpatient episodes.
